SEQUENCE LISTING (6 sequences)

SEQ ID NO: 1 - protein, 45 aa, Artificial Sequence
  Description of Artificial Sequence Synthetic polypeptide
GGGGGGGGGGGGGGGGGGGGRRRRRRRRRRRRRRRRRRRRRRRRR

SEQ ID NO: 2 - protein, 51 aa, Artificial Sequence
  Description of Artificial Sequence Synthetic polypeptide
GGGGGGGGGGGGGGGGGGGGRGRDDRRQRRRGGGGGGGGGGGGGGGGGGGG

SEQ ID NO: 3 - protein, 51 aa, Artificial Sequence
  Description of Artificial Sequence Synthetic polypeptide
GGGGGGGGGGGGGGGGGGGGYGRKKRRQRRRGGGGGGGGGGGGGGGGGGGG

SEQ ID NO: 4 - protein, 49 aa, Artificial Sequence
  Description of Artificial Sequence Synthetic polypeptide
GGGGGGGGGGGGGGGGGGGGRKKRRQRRRGGGGGGGGGGGGGGGGGGGG

SEQ ID NO: 5 - protein, 35 aa, Artificial Sequence
  Description of Artificial Sequence Synthetic polypeptide
RKKRRQRRRGKKKKKKKKKKKKKKKGRKKRRQRRR

SEQ ID NO: 6 - protein, 10 aa, Artificial Sequence
  Description of Artificial Sequence Synthetic peptide
GGGRRRRRRR

(19) United States
(12) Patent Application Publication
(10) Pub. No.: US 2011/0106021 A1
(43) Pub. Date: May 5, 2011

(54) Device and Method for Topical Application of Therapeutics or Cosmetic Compositions

(75) Inventors: Curtis Ruegg, Redwood City, CA (US); David Reynolds, Bromont (CA); Daniel MacDonald, Bromont (CA); Yan Tremblay, Orford (CA)

(73) Assignees: Revance Therapeutics, Inc., Newark, CA (US); Duoject Medical Systems, Inc., Bromont (CA)

(21) Appl. No.: 12/911,398

(22) Filed: Oct. 25, 2010

Related U.S. Application Data
(60) Provisional application No. 61/256,837, filed on Oct. 30, 2009.
(60) Provisional application No. 61/280,169, filed on Oct. 30, 2009.

(51) Int. Cl. A61M 35/00

(52) U.S. Cl. 604/290; 604/310

(57) ABSTRACT

This invention relates to devices and methods for safely reconstituting and administering topical therapeutic or cosmetic compositions. The topical applicator according to the invention is particularly well suited for storing, reconstituting, and administering or applying highly toxic substances.

This application claims the benefit of priority to U.S. Provisional Application No. 61/256,837, filed on Oct. 30, 2009. This application also claims the benefit of priority to a U.S. Provisional Application No. 61/280,169 entitled “Inter Vial Transfer System,” also filed on Oct. 30, 2009. The contents of these two provisional applications are hereby incorporated by reference in their entirety.

### FIELD OF THE INVENTION

This invention relates to devices and methods for safely reconstituting and administering topical therapeutic or cosmetic compositions.

### BACKGROUND OF THE INVENTION

Many therapeutic or cosmetic compositions are distributed and stored as two or more separate components, which are mixed just prior to administration. For example, a vaccine may be distributed as a lyophilized powder that is reconstituted with a diluent just prior to injection. Reconstitution of an injectable composition usually involves drawing a liquid diluent into a syringe, inserting the needle of the syringe through a penetrable seal of a vial containing a lyophilized active ingredient, and then injecting the diluent into the vial. After the injectable composition is fully reconstituted, it is drawn into the syringe. The needle of the syringe is then withdrawn from the vial and inserted into a subject in need of the therapeutic or cosmetic agent contained in the injectable composition.

With recent advances in transdermal carrier technology, it has become possible to achieve topical administration of therapeutic or cosmetic agents that previously could only be administered by injection. (see e.g., U.S. patent application Ser. Nos. 09/910,432; 11/072,026; and 11/073,307, which are hereby incorporated by reference in their entirety). Like injectable compositions, topical compositions may distributed and stored as two or more separate components, which are mixed just prior to administration. However, reconstituting and administering topical compositions may be complicated by the fact that, at least in some instances, a syringe with a needle may not be an appropriate device for adding diluent to a lyophilized active ingredient. For example, a topical formulation may contain a concentration of the active agent that is higher than the maximum allowable concentration for an injectable formulation. Accordingly, reconstitution of such a topical formulation with a syringe having a needle could lead to accidental overdose of the active agent, if the clinician, in a moment of confusion, mistakenly injects the topical formulation after reconstitution with a syringe.

Botulinum toxin is an example of a therapeutic or cosmetic agent that can be administered by either injection or topical administration. See e.g., U.S. patent application Ser. No. 11/072,026. Botulinum toxin (also known as botulin toxin or botulinum neurotoxin) is a neurotoxin produced by the gram-positive bacteria Clostridium botulinum. It produces paralysis of muscles by preventing synaptic transmission or release of acetylcholine across the neuromuscular junction, and is thought to act in other ways as well. Botulinum toxin essentially blocks signals that normally would cause muscle spasms or contractions, resulting in paralysis. These properties of botulinum toxin have been used to treat a variety of conditions, including hemifacial spasm, adult onset spasmodic torticollis, anal fissure, blepharospasm, cerebral palsy, cervical dystonia, migraine headaches, strabismus, temperomandibular joint disorder, and various types of muscle cramping and spasms. More recently, the muscle-paralyzing effects of botulinum toxin have been taken advantage of in therapeutic and cosmetic facial applications such as treatment of wrinkles, frown lines, and other results of spasms or contractions of facial muscles.

Botulinum toxin type A, which is one of eight serologically related naturally occurring botulinum toxins, is said to be the most lethal natural biological agent known to man. Nonetheless, the muscle-paralyzing effects of botulinum toxin type A have been used for a variety of therapeutic and cosmetic purposes. Conventionally, botulinum toxin type A is administered via injection to the area in need of treatment. Botulinum toxin type A is commercially available as a lyophilized mixture of botulinum toxin and various stabilizing agents, such as albumin. Immediately prior to administration, the botulinum toxin is reconstituted by introducing a liquid diluent, typically saline, via a syringe into the vial containing the lyophilized botulinum toxin. The reconstituted mixture is then drawn into the syringe. This sequence of steps is relatively safe for the clinician, because the botulinum toxin mixture is contained in either the syringe or the vial until administration of the injectable botulinum toxin composition.

However, due to the extreme toxicity of botulinum toxin, reconstitution of lyophilized topical formulations of botulinum toxin using syringe with a needle may be dangerous. For instance, a topical botulinum toxin formulation may contain a toxin concentration that is much higher than the maximum acceptable level for an injectable botulinum toxin formulation. Accordingly, reconstitution of topical botulinum toxin formulations with a syringe could lead to accidental fatal overdose of botulinum toxin, if the reconstituted topical formulation is inadvertently injected into a patient.

Thus, there exists a need for improved devices and methods that can permit the safe reconstitution and administration of topical compositions comprising therapeutic or cosmetic agents.

### SUMMARY OF THE INVENTION

This invention provides devices and methods for safely reconstituting topical compositions comprising therapeutic or cosmetic active agents. The devices and methods disclosed herein permit the safe reconstitution of a variety of topical compositions, including those in which the therapeutic or cosmetic active agent is difficult to handle, due to toxicity, susceptibility towards decomposition, or other reasons.

One object of the invention is to provide a topical applicator for topical administration of a paralytic agent that acts as a therapeutic or cosmetic agent. The applicator includes a vial socket for receiving a vial that contains a solid therapeutic or cosmetic composition that is sealed within the vial by a penetrable seal. In certain preferred embodiments, the solid therapeutic or cosmetic composition comprises a paralytic agent, as defined herein. The vial socket is configured to prevent removal of the vial from the vial socket once the vial is inserted into the vial socket. The applicator further includes a cartridge with an adjustable volume for housing a diluent to reconstitute the solid therapeutic or cosmetic composition. The cartridge comprises a first end with a penetrable seal and a second end with a plunger sealing an open end thereof. The applicator also includes a housing with a first end and a second end, the first end comprising a dispensing tip with an orifice and further being adapted to be releasably connected to the vial socket, and the second end being adapted to receive the cartridge. The housing also comprises a needle hub located between the cartridge and the vial socket, the needle hub comprising a first transfer needle that points towards the penetrable seal of the vial and a second transfer needle that points towards the penetrable seal of the cartridge. Additionally, the topical applicator includes an activation cap that that is configured to be inserted into the second end of the cartridge socket in order to drive the cartridge and the needle hub towards the vial socket. Thus, when a vial is held in the vial socket, the first and second transfer needles pierce the penetrable seals of the vial and cartridge, respectively, thereby fluidly connecting the vial to the cartridge. The applicator also includes a slidable plunger rod that is attached to the slidable sealing member of the cartridge. The slideable plunger rod is configured to force the diluent into the vial containing the solid therapeutic or cosmetic composition thereby forming a reconstituted topical composition. The slidable plunger rod is also configured to withdraw the reconstituted topical composition into the cartridge. Once the reconstituted composition is drawn into the cartridge, the vial socket and vial are detached from the topical applicator, thereby exposing a dispensing member. In preferred embodiments, the dispensing member is configured such that it does not accept needles, thereby preventing inadvertent injection of the reconstituted topical composition.

Another object of the invention is to provide a topical applicator for a paralytic agent, wherein the topical applicator comprises a housing having first and second open ends, with the first open end configured to be releasably connected to a vial socket that is adapted to receive a vial containing a solid therapeutic or cosmetic agent to be reconstituted, and the second end adapted to receive a cartridge. The cartridge has a plunger that seals an open end thereof and a septum located at an end of the cartridge opposite the plunger. The cartridge is loaded with a paralytic agent and a diluent that reconstitutes the paralytic agent. The topical applicator further comprises a needle hub mounted within the housing, where the needle hub has a needle having first and second piercing ends mounted in the needle hub. One of the piercing ends of the needle hub establishes a fluid connection between the contents of the cartridge and a dispensing member that is attached to the distal end of the housing. The topical applicator also comprises an activation cap for causing the needle to penetrate a septum of a vial (when a vial is held in the vial socket) and the septum of the cartridge to permit transfer of components therebetween.

Yet another aspect of the invention is to provide a method for topically applying a paralytic agent. The method comprises pushing the activation cap of the topical applicator as described herein, to cause the needle to penetrate a septum of a vial held in the vial socket and the septum of the cartridge, thereby establishing a fluid connection. The method also comprises pushing on the plunger to force the diluent into the vial and optionally shaking the vial, thereby forming a reconstituted paralytic composition, and then withdrawing the plunger to cause the reconstituted paralytic composition to be drawn into the cartridge. The method also includes removing the vial socket and vial to expose a dispensing member, and pushing on the plunger to cause the reconstituted paralytic composition to be dispensed from the dispensing member onto an area on a patient in need of treatment.

### BRIEF DESCRIPTION OF THE DRAWINGS

FIG. 1 is an exploded view of a topical applicator according to an embodiment of the present invention.

FIG. 2 is an enlarged exploded view of the topical applicator of FIG. 1 with dash lines showing internal structure of certain components.

FIG. 3 is a cross-sectional view of a topical applicator prior to activation.

FIGS. 4 to 6 are sectional views illustrating operation of the topical applicator.

FIG. 7 is a sectional view illustrating detachment of the vial socket from the cartridge portion.

FIG. 8 is a perspective of the cartridge and housing with the composition ready for application.

FIGS. 9 and 10 show different applicator tips.

### DETAILED DESCRIPTION OF THE INVENTION

This invention provides devices and methods for safely reconstituting solid compositions for topical administration. In preferred embodiments, the devices and methods according to the invention permit reconstitution of solid compositions stored in a vial, without the use of a syringe with an exposed needle. In this way, the possibility of inadvertently injecting into a patient a topical composition not formulated for injection is minimized. In addition, in certain preferred embodiments of the invention, the topical applicator is configured to prevent the easy removal of the vial, once the vial is attached to the topical applicator, in order to prevent the user from attempting to complete the reconstitution process with a syringe equipped with a needle.

### Topical Applicator

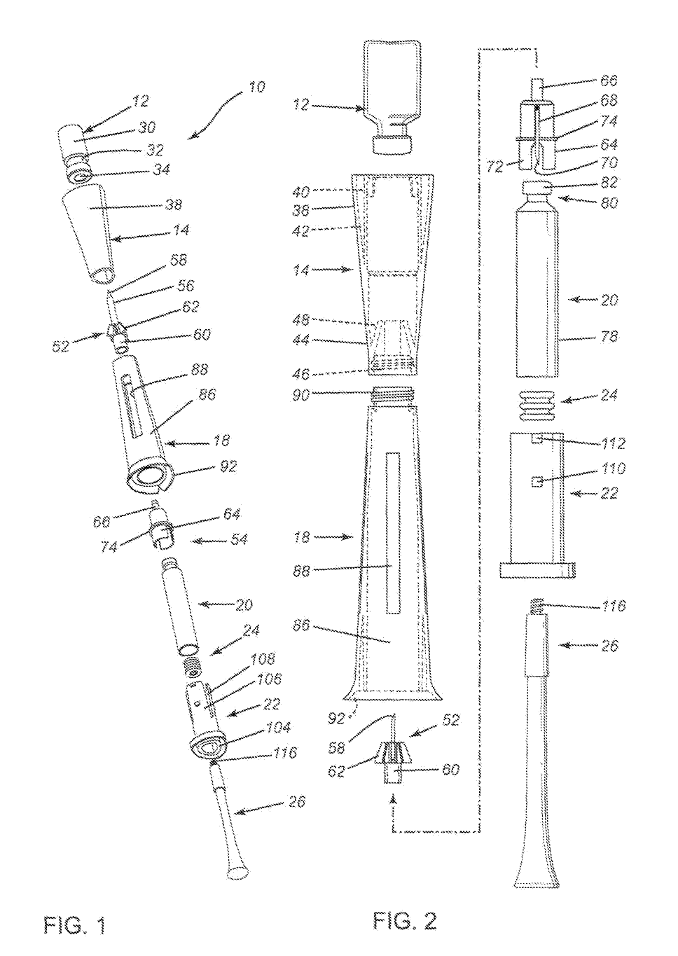

FIG. 1 shows a topical applicator 10 according to an exemplary embodiment of the invention. In describing various components, the terms “proximal” and “distal” are utilized. In each instance, the term proximal refers to the end closest to the hand of the user while the term distal refers to the end furthest removed from the hand of the user.

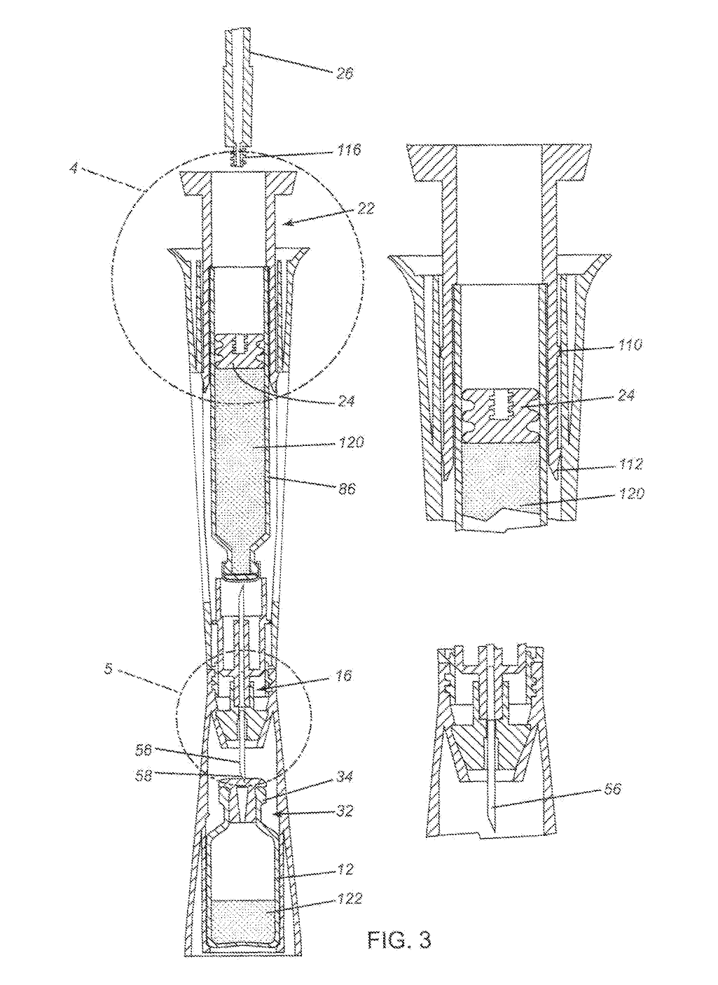

A vial generally designated by reference numeral 12 is associated with the topical applicator that also includes a vial socket 14 designed to receive vial 12. Topical applicator 10 also includes a needle hub generally designated by reference numeral 16 (FIG. 3). A housing 18 is designed to extend about a cartridge 20. The proximal end of topical applicator 10 includes an activation cap 22. A plunger 24 is designed to fit within the open end cartridge 20 while a plunger rod 26 is engageable with plunger 24 as will be discussed hereinbelow.

Vial 12 may be any conventional vial known to those skilled in the art or alternatively, in certain applications, may be of a non standard size when it is desired to use some specialized components for the vial. Vial 12 will include a body portion 30 having a restricted neck portion 32 over which extends a pierceable septum 34. In preferred embodiments, the material from which vial 12 is made does not significantly degrade or denature the dried active agent either prior to or during reconstitution.

As shown in FIG. 1, vial socket 14 is configured to receive vial 12 at the distal end of vial socket 14. Vial socket 14 is, in this illustrative embodiment, of a somewhat overall triangular configuration having a plurality of lower outer wall segments 38 each of which is somewhat arcuate in configuration and tapers inwardly from a distal end to meet upper wall segments 44. Lower wall segments 38 define the lower body and there are provided a plurality of inner legs 40 each having inwardly extending flanges for gripping vial 12 at their distal end and being spaced from the wall by means of ribs 42 which extend between inner legs 40 and lower outer wall segments 38.

Vial socket 14 also includes upper wall segments 44 which define, at a proximal end thereof, a female thread opening 46. A plurality of flanges 48 extend downwardly as may be seen in FIG. 2.

Needle hub 16 comprises a distal member 52 and a proximal member 54 which are designed to fit together. Distal member 52 includes a piercing member 56 having a piercing tip 58. In preferred embodiments, the gauge of the piercing member 56 is in the range of 18-25 gauge, in the range of 18-23 gauge, or in the range of 18-21 gauge. A suitable gauge can be readily determined by one of ordinary skill in the art based on the consideration of the viscosity of the topical composition, toxicity, and other factors. At its proximal end, distal member 52 has a tubular end 60. A plurality of pins 62 extend circumferentially of distal member 52.

Proximal member 54 includes a body portion 64 having a tubular portion 66 which is designed to engage with tubular end 60 of distal member 52. A piercing member 68 is secured to body portion 64 and has a piercing tip 70.

Proximal member 54 also includes a pair of legs 72 with an annular ring 74 situated proximate the middle of body 64.

Cartridge 20 includes a body 78 which has an open end designed to receive plunger 24. A pierceable septum 82 is arranged at the top of body 78 adjacent neck 80. Housing 18, in the illustrated embodiment, includes a plurality of wall segments 86, there being three such wall segments 86 in the illustrated embodiment. In each wall segment 86 there is provided a slot 88 to provide visual access to the interior. Housing 18 also includes a plurality of male threads 90 at the distal end thereof. Housing 18 also has a flared proximal end 92.

Activation cap 22 has a proximal end wall 104 and a side wall 106. A first set of protrusions 110 are designed to engage housing 18 when the activation cap has been activated while a second set of protrusions 112 engage housing 18 prior to activation.

Plunger rod 26 is provided with male threads 116 for screwthreadebly engaging plunger 24.

In operation, vial 12 and vial socket 14 are supplied as a unit with the vial inserted therein and retained in a non removable manner. Similarly, cartridge 20 is mounted within housing 18 and activation cap 22 inserted in the proximal end of housing 18. Activation cap 18 is held in a non removable position. Housing 18 is screwthreadably engaged with vial socket 14 by means of respective threads 90, 46.

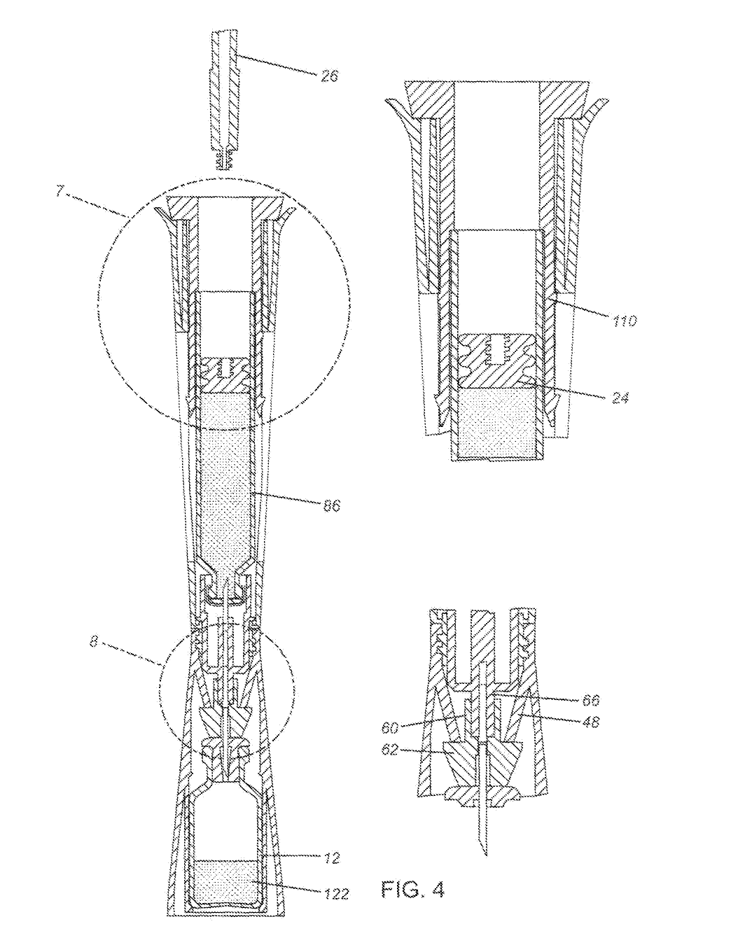

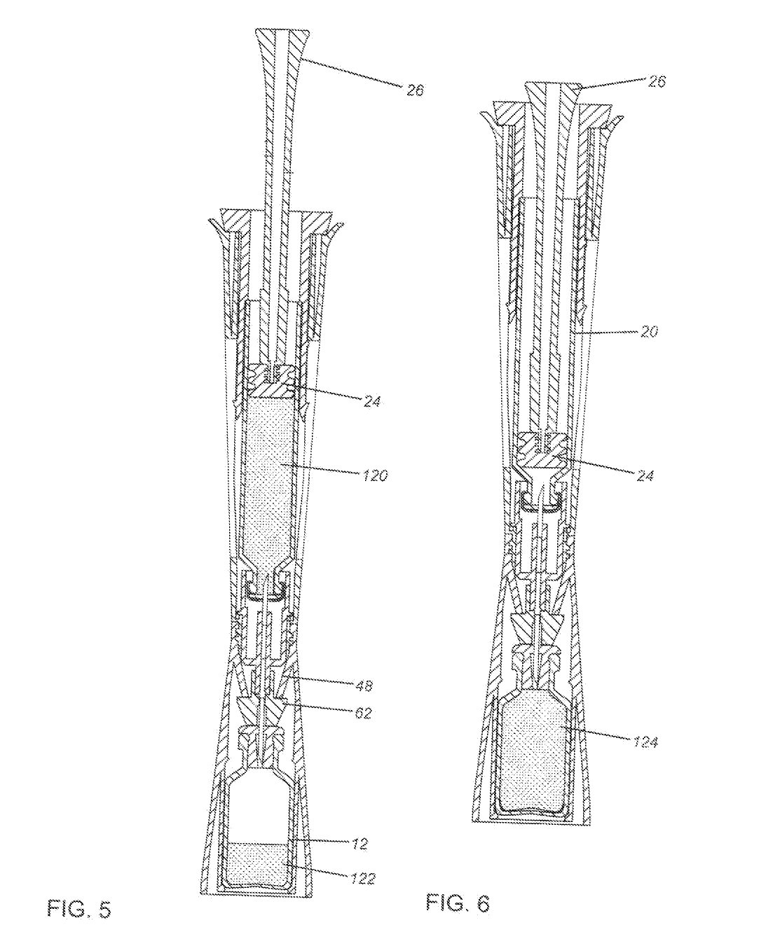

As illustrated in FIG. 3, activation cap 22 extends exteriorly of housing 18. For use, activation cap 22 is depressed as shown in FIGS. 4 and 5 thereby leading to a piercing of septum 34 of vial 12 and septum 82 of cartridge 20. Plunger rod 26 is then engaged with plunger 24 by means of their respective screwthreads and pressure is exerted on plunger 24 to transfer the diluent 120 to mix with a component 122 in vial 12. This position is illustrated in FIG. 6.

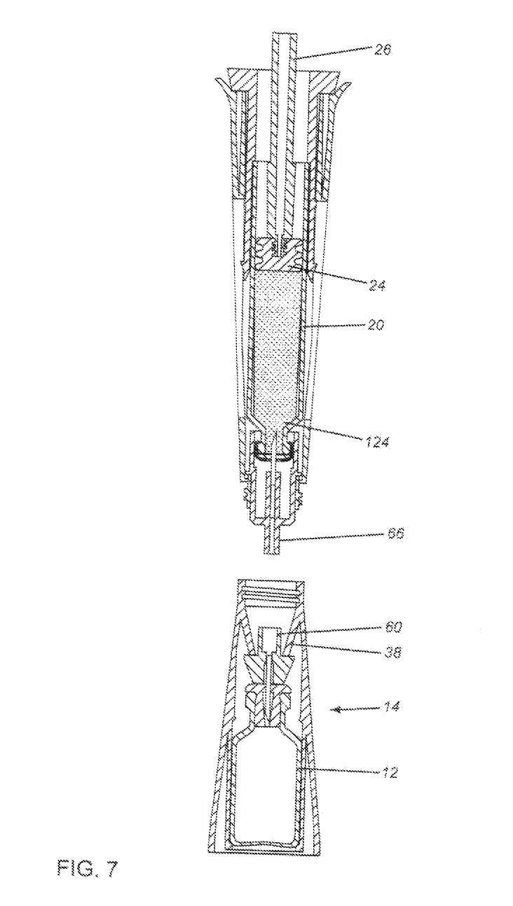

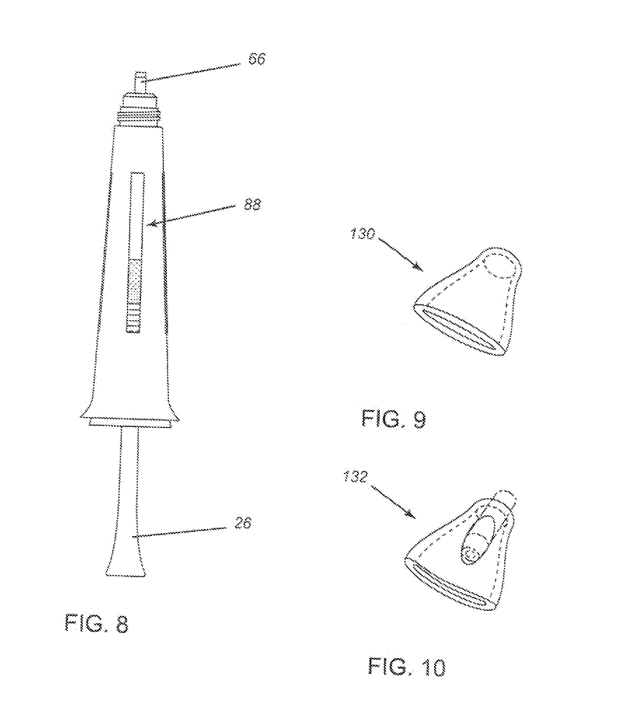

At this point in time, a gentle shaking of the vial 12 may occur to ensure mixing of the components, subsequently the mixture 122 is aspirated into cartridge 20 as shown in FIG. 7. The housing is then removed from vial socket 14 and the mixture 124 is then dispensed as required. In the illustrated embodiment, tubular portion 66 forms the dispensing member and is specifically designed to apply mixture 124 in a topical manner. To ensure that the mixture is not injected, member 66 may be of a non standard size and/or configuration not designed to accepted a needle. However, in certain applications, the attachment of a needle may be desired and appropriate configurations would be provided. In certain preferred embodiments, dispensing member 66 comprises an orifice having a diameter that ranges from 18-25 gauge, more preferably from 18-23 gauge, and most preferably from 18-21 gauge. A suitable choice of the diameter can be readily determined by one of ordinary skill in the art based on the consideration of the viscosity of the topical composition, toxicity, and other factors.

FIGS. 9 and 10 illustrate different dispensing tips which may be utilized for topical applications.

### Therapeutic and Cosmetic Compositions

The therapeutic or cosmetic topical compositions that are dispensed by the topical applicator according to the invention are not particularly limited, and may comprise any active agent capable of producing a therapeutic or cosmetic benefit after being topically applied to a surface region of a subject's body. The topical applicator according to the invention is particularly well suited for storing, reconstituting, and administering or applying highly toxic substances. Non-limiting examples of active agents that are suitable for the topical therapeutic compositions according to the invention include analgesic agents, anti-asthmatic agents, antibiotics, antidepressant agents, anti-diabetic agents, antifungal agents, antiemetics, antihypertensives, anti-impotence agents, anti-inflammatory agents, antineoplastic agents, anti-HIV agents, antiviral agents, anxiolytic agents, contraception agents, fertility agents, antithrombotic agents, prothrombotic agents, hormones, vaccines, immunosuppressive agents, vitamins and the like. Non-limiting examples of suitable cosmetic agents include, for example, epidermal growth factor (EGF), as well as human growth hormone, antioxidants, and botulinum toxin. In certain particularly preferred embodiments, the topical compositions according to the invention comprise insulin, botulinum toxin, VEGF, EGF, antibodies to VEGF, or TGF-β1.

Generally speaking, the topical applicator according to the invention may be used for topical administration of a therapeutic or cosmetic formulation to any region of the body in need thereof. In certain preferred embodiments, the area of the body to be treated is selected from the group consisting of the face, the axilla, the palms of the hands, the hands, the feet, the lower back, the neck, the leg, the groin, the arm, the elbow, the knee, the pelvis, the buttocks and the torso.

In certain preferred embodiments, the topical applicator according to the invention is used to administer a topical composition that comprises a paralytic agent. Generally speaking, a paralytic agent may be any agent that can interrupt nerve impulse transmission across a neuromuscular or neuroglandular junction, block or reduce neuronal exocytosis of a neurotransmitter or alter the action potential at a sodium channel voltage gate of a neuron. Non-limiting examples of paralytic substances contemplated by the invention include botulinum toxins (including serotypes A, B, C₁, D, E, F, and G), tetanus toxins, saxitoxins, and tetrodotoxin, as well as combinations thereof.

In some embodiments, the paralytic substance is topically administered to produce a cosmetic effect. For instance, the paralytic substance, (such as, e.g., botulinum toxin type A neurotoxin) may be applied to the face to reduce the appearance of wrinkles, including marionette lines, nasolabial lines, crows feet, brow furrows, glabellar lines, and combinations thereof.

In other embodiments, the paralytic substance is topically administered to provide a therapeutic effect to a subject. For instance, the paralytic substance may be a substance capable of attenuating cholinergic nerve impulses, thereby suppressing output from a gland. In certain non-limiting embodiments, the paralytic substance comprises botulinum toxin that is administered to reduce hypersecretion of sweat glands or sebaceous glands, in order to treat hyperhidrosis or acne, respectively. More generally, this invention also contemplates the administration of topical botulinum toxin compositions using the topical applicator of the invention to treat any indication for which botulinum toxin is known to provide an improvement in condition. Non-limiting examples of such indications include hemifacial spasm, adult onset spasmodic torticollis, anal fissure, blepharospasm, cerebral palsy, cervical dystonia, migraine headaches, strabismus, temperomandibular joint disorder, and various types of muscle cramping and spasms.

In certain particularly preferred embodiments, the topical compositions to be applied by the topical applicator according to the invention comprise a botulinum toxin. The term “botulinum toxin” as used herein is meant to refer to any of the known types of botulinum toxin, whether produced by the bacterium or by recombinant techniques, as well as any such types that may be subsequently discovered including engineered variants or fusion proteins. The botulinum toxin may be obtained from any of the known serotypes of C. botulinum (e.g., serotypes A, B, C₁, D, E, F, or G). In certain preferred embodiments, the botulinum toxin is present as an isolated botulinum toxin molecule (e.g., botulinum toxin type A neurotoxin) that has been stabilized by exogenous stabilizers. See, e.g., U.S. Provisional Application No. 61/220,433 entitled “Albumin Free Botulinum Toxin Formulations,” which is hereby incorporated by reference in its entirety. Alternatively, the botulinum toxin may be present in a complexed form, stabilized, at least in part, by one or more of the non-toxin, non-hemaglutinin proteins and non-toxin, hemaglutinin proteins that are normally expressed along with the botulinum toxin by the C. Botulinum bacteria. In certain embodiments, the botulinum toxin is stabilized by exogenous stabilizers, such as albumin. The invention also specifically contemplates the use of the topical applicator to reconstituted and administer commercially available botulinum toxin formulations, non-limiting examples of which include BOTOX™, Dysport™, and Xeomin™

The botulinum toxin used in the applicator of this invention can alternatively be a botulinum toxin derivative, that is, a compound that has botulinum toxin activity but contains one or more chemical or functional alterations on any part or on any chain relative to naturally occurring or recombinant native botulinum toxins. For instance, the botulinum toxin may be a modified neurotoxin (e.g., a neurotoxin which has at least one of its amino acids deleted, modified or replaced, as compared to a native, or a recombinantly produced neurotoxin or a derivative or fragment thereof). For instance, the botulinum toxin may be one that has been modified in a way that, for instance, enhances its properties or decreases undesirable side effects, but that still retains the desired botulinum toxin activity. The botulinum toxin may be any of the botulinum toxin complexes produced by the bacterium, as described above. Alternatively, the botulinum toxin may be a toxin prepared using recombinant or synthetic chemical techniques (e.g. a recombinant peptide, a fusion protein, or a hybrid neurotoxin, as prepared from subunits or domains of different botulinum toxin serotypes (see U.S. Pat. No. 6,444,209, for instance)). The botulinum toxin may also be a portion of the overall molecule that has been shown to possess the necessary botulinum toxin activity, and in such case may be used per se or as part of a combination or conjugate molecule, for instance a fusion protein. Additionally, the botulinum toxin may be in the form of a botulinum toxin precursor, which may itself be non-toxic, for instance a nontoxic zinc protease that becomes toxic on proteolytic cleavage.

The therapeutic or cosmetic compositions contemplated by the invention are typically stored in solid form, using the methods described herein. For example, the compositions may be lyophilized into a powder that can be stored in a vial for an extended period of time before being reconstituted by a diluent.

The diluent for reconstituting the therapeutic or cosmetic compositions of the invention include any pharmaceutically or cosmetically acceptable diluent that is capable of reconstituting the solid therapeutic or cosmetic composition. In certain embodiments, the diluent is simply water, saline, or a pharmaceutically acceptable buffer. Non-limiting examples of such buffers include those involving salts of citric acid, acetic acid, succinnic acid, tartaric acid, maleic acid, and histidine. Non-limiting examples of suitable buffer concentrations include buffer concentrations in the range of 0.400% to 0.600%; 0.450% to 0.575%, or 0.500% to 0.565%. The invention also contemplates diluents comprising a mixture of buffer salts, non-limiting examples of which include citrate/acetate, citrate/histidine, citrate/tartrate, maleate/histidine, or succinate/histidine. In certain preferred embodiments, the buffer is phosphate buffer.

In some preferred embodiments, the diluent also contains a viscosity modifying agent that is capable of increasing the viscosity of the composition, so as to make the topical application of the composition easier and more accurate. For instance, the viscosity modifying agent may be a gelling agent. The viscosity modifying agent may be chosen to prevent the reconstituted composition from drying out, which can cause a decrease in the activity of the certain active agents, such as botulinum toxin. Particularly preferred viscosity modifying agents are those that are uncharged and do not interfere with the activity of the active agent or the efficiency of the penetration of the topical compositions upon administration. The viscosity modifying agents may contain cellulose-based gelling agents, a non-limiting example of which is a hydroxylalkylcellulose, such as hydroxypropylcellulose (HPC) or hydroxypropyl methylcellulose. In certain preferred embodiments, the therapeutic or cosmetic compositions comprise 2-4% HPC. Alternatively, the viscosity modifying agent may be a polyalcohol, a non-limiting example of which is polyethylene glycol (PEG).

In some embodiments, the diluent comprises a poloxamer, non-limiting examples of which include 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, and poloxamer 407. In certain preferred embodiments, the poloxamer is present in a concentration that ranges from 10-30%, or from 13-25%, or from 14-21%, or from 15-17%, or even from 16-16.5%.

In certain preferred embodiments, the topical compositions according to the invention further comprise a carrier that promotes transdermal transport of the therapeutic or cosmetic active agent. For example, the carrier may be a positively charged carrier molecule with positively charged efficiency groups attached thereto. In certain preferred embodiments, the topical transport is enhanced by the carrier without covalent modification of the therapeutic or cosmetic active agent to be delivered.

By “positively charged” is meant that the carrier has a positive charge under at least some solution-phase conditions, more preferably under at least some physiologically compatible conditions. More specifically, “positively charged” as used herein, means that the group in question contains functionalities that are charged under all pH conditions, for instance, a quaternary amine, or contains a functionality which can acquire positive charge under certain solution-phase conditions, such as pH changes in the case of primary amines. More preferably, “positively charged” as used herein refers to those groups that have the behavior of associating with anions over physiologically compatible conditions. Polymers with a multiplicity of positively-charged moieties need not be homopolymers, as will be apparent to one skilled in the art. Other examples of positively charged moieties are well known in the prior art and can be employed readily, as will be apparent to those skilled in the art.

Generally, the positively-charged carrier comprises a “positively charged backbone,” which is typically a linear chain of atoms, either with groups in the chain carrying a positive charge at physiological pH, or with groups carrying a positive charge attached to side chains extending from the backbone. Preferably, the positively charged backbone itself will not have a defined enzymatic or therapeutic biologic activity. The linear backbone is a hydrocarbon backbone which is, in some embodiments, interrupted by heteroatoms selected from nitrogen, oxygen, sulfur, silicon and phosphorus. The majority of backbone chain atoms are usually carbon. Additionally, the backbone will often be a polymer of repeating units (e.g., amino acids, poly(ethyleneoxy), poly(propyleneamine), polyalkyleneimine, and the like) but can be a heteropolymer. In one group of embodiments, the positively charged backbone is a polypropyleneamine wherein a number of the amine nitrogen atoms are present as ammonium groups (tetra-substituted) carrying a positive charge. In another embodiment, the positively charged backbone is a nonpeptidyl polymer, which may be a hetero- or homo-polymer such as a polyalkyleneimine, for example a polyethyleneimine or polypropyleneimine, having a molecular weight of from about 10,000 to about 2,500,000, preferably from about 100,000 to about 1,800,000, and most preferably from about 500,000 to about 1,400,000. In another group of embodiments, the backbone has attached a plurality of side-chain moieties that include positively charged groups (e.g., ammonium groups, pyridinium groups, phosphonium groups, sulfonium groups, guanidinium groups, or amidinium groups). The sidechain moieties in this group of embodiments can be placed at spacings along the backbone that are consistent in separations or variable. Additionally, the length of the sidechains can be similar or dissimilar. For example, in one group of embodiments, the sidechains can be linear or branched hydrocarbon chains having from one to twenty carbon atoms and terminating at the distal end (away from the backbone) in one of the above-noted positively charged groups. In all aspects of the present invention, the association between the carrier and the therapeutic or cosmetic active agent is by non-covalent interaction, non-limiting examples of which include ionic interactions, hydrogen bonding, van der Waals forces, or combinations thereof.

In one group of embodiments, the positively charged backbone is a polypeptide having multiple positively charged sidechain groups (e.g., lysine, arginine, ornithine, homoarginine, and the like). Preferably, the polypeptide has a molecular weight of from about 10,000 to about 1,500,000, more preferably from about 25,000 to about 1,200,000, most preferably from about 100,000 to about 1,000,000. One of skill in the art will appreciate that when amino acids are used in this portion of the invention, the sidechains can have either the D- or L-form (R or S configuration) at the center of attachment. Alternatively, the backbone can be an analog of a polypeptide such as a peptoid. See, for example, Kessler, Angew. Chem. Int. Ed. Engl. 32:543 (1993); Zuckermann et al. Chemtracts-Macromol. Chem. 4:80 (1992); and Simon et al. Proc. Nat'l. Acad. Sci. USA 89:9367 (1992)). Briefly, a peptoid is a polyglycine in which the sidechain is attached to the backbone nitrogen atoms rather than the α-carbon atoms. As above, a portion of the sidechains will typically terminate in a positively charged group to provide a positively charged backbone component. Synthesis of peptoids is described in, for example, U.S. Pat. No. 5,877,278, which is hereby incorporated by reference in its entirety. As the term is used herein, positively charged backbones that have a peptoid backbone construction are considered “non-peptide” as they are not composed of amino acids having naturally occurring sidechains at the α-carbon locations.

A variety of other backbones can be used employing, for example, steric or electronic mimics of polypeptides wherein the amide linkages of the peptide are replaced with surrogates such as ester linkages, thioamides (—CSNH—), reversed thioamide (—NHCS—), aminomethylene (—NHCH₂—) or the reversed methyleneamino (—CH₂NH—) groups, keto-methylene (—COCH₂—) groups, phosphinate (—PO₂RCH₂—), phosphonamidate and phosphonamidate ester (—PO₂RNH—), reverse peptide (—NHCO—), trans-alkene (—CR═CH—), fluoroalkene (—CF═CH—), dimethylene (—CH₂CH₂—), thioether (—CH₂S—), hydroxyethylene (—CH(OH)CH₂—), methyleneoxy (—CH₂O—), tetrazole (CN₄), sulfonamido (—SO₂NH—), methylenesulfonamido (—CHRSO₂NH—), reversed sulfonamide (—NHSO₂—), and backbones with malonate and/or gem-diamino-alkyl subunits, for example, as reviewed by Fletcher et al. ((1998) Chem. Rev. 98:763) and detailed by references cited therein. Many of the foregoing substitutions result in approximately isosteric polymer backbones relative to backbones formed from α-amino acids.

In each of the backbones provided above, sidechain groups can be appended that carry a positively charged group. For example, the sulfonamide-linked backbones (—SO₂NH— and —NHSO₂—) can have sidechain groups attached to the nitrogen atoms. Similarly, the hydroxyethylene (—CH(OH)CH₂—) linkage can bear a sidechain group attached to the hydroxy substituent. One of skill in the art can readily adapt the other linkage chemistries to provide positively charged sidechain groups using standard synthetic methods.

In one embodiment, the positively charged backbone is a polypeptide having efficiency groups. As used herein, an efficiency group is any agent that has the effect of promoting the translocation of the positively charged backbone through a tissue or cell membrane. Non-limiting examples of efficiency groups include -(gly)ₙ₁-(arg)ₙ₂, HIV-TAT or fragments thereof, or the protein transduction domain of Antennapedia, or a fragment thereof, in which the subscript n1 is an integer of from 0 to 20, more preferably 0 to 8, still more preferably 2 to 5, and the subscript n2 is independently an odd integer of from about 5 to about 25, more preferably about 7 to about 17, most preferably about 7 to about 13. Still further preferred are those embodiments in which the HIV-TAT fragment has the formula (gly)ₚ-RGRDDRRQRRR-(gly)_{q}, (gly)ₚ-YGRKKRRQRRR-(gly)_{q} or (gly)ₚ-RKKRRQRRR-(gly)_{q} wherein the subscripts p and q are each independently an integer of from 0 to 20 and the fragment is attached to the backbone via either the C-terminus or the N-terminus of the fragment. Preferred HIV-TAT fragments are those in which the subscripts p and q are each independently integers of from 0 to 8, more preferably 2 to 5. In some embodiments, the carrier has the amino acid sequence RKKRRQRRR-G-(K)₁₅-G-RKKRRQRRR.

In another preferred embodiment the positively charged efficiency group is the Antennapedia (Antp) protein transduction domain (PTD), or a fragment thereof that retains activity. (See, e.g., Console et al., J. Biol. Chem. 278:35109 (2003), the contents of which are incorporated by reference in their entirety.) Preferably the positively charged carrier includes side-chain positively charged efficiency groups in an amount of at least about 0.05%, as a percentage of the total carrier weight, preferably from about 0.05 to about 45 weight %, and most preferably from about 0.1 to about 30 weight %. For positively charged efficiency groups having the formula -(gly)ₙ₁-(arg)ₙ₂, the most preferred amount is from about 0.1 to about 25%.

In another embodiment, the backbone portion is a polylysine and positively charged efficiency groups are attached to the lysine sidechain amino groups. In some embodiments, the polylysine may have a molecular weight that ranges from about 10,000 to about 1,500,000, preferably from about 25,000 to about 1,200,000, and most preferably from about 100,000 to about 1,000,000. In other embodiments, the polylysine may have a molecular weight that ranges from about 500 to about 5000. about 1000 to about 4000, about 1500 to about 3500, or about 2000 to about 3000. The polylysine may be any of the commercially available (Sigma Chemical Company, St. Louis, Mo., USA) polylysines such as, for example, polylysine having MW>70,000, polylysine having MW of 70,000 to 150,000, polylysine having MW 150,000 to 300,000 and polylysine having MW>300,000. The selection of an appropriate polylysine will depend on the remaining components of the composition and will be sufficient to provide an overall net positive charge to the composition and, in some embodiments, provide a length that is preferably from one to four times the combined length of the negatively charged components. Preferred positively charged efficiency groups or efficiency groups include, for example, -gly-gly-gly-arg-arg-arg-arg-arg-arg-arg (-Gly₃Arg₇) or HIV-TAT. In another preferred embodiment the positively charged backbone is a long chain polyalkyleneimine such as a polyethyleneimine, for example, one having a molecular weight of about 1,000,000.

In another embodiment, the carrier is a polylysine with positively charged branching groups attached to the lysine side-chain amino groups. The polylysine used in this particularly embodiment can be any of the commercially available (Sigma Chemical Company, St. Louis, Mo., USA, e.g.) polylysines such as, for example, polylysine having MW>70,000, polylysine having MW of 70,000 to 150,000, polylysine having MW 150,000 to 300,000 and polylysine having MW>300,000. However, preferably the polylysine has MW of at least about 10,000. Preferred positively charged branching groups or efficiency groups include, for example, -gly-gly-gly-arg-arg-arg-arg-arg-arg-arg (-Gly₃Arg₇), HIV-TAT or fragments of it, and Antennapedia PTD or fragments thereof.

In other embodiments of this invention, the carrier is a relatively short polylysine or polyethyleneimine (PEI) backbone (which may be linear or branched) and which has positively charged branching groups. Such carriers are useful for minimizing uncontrolled aggregation of the backbones and botulinum toxin in a therapeutic composition, which causes the transport efficiency to decrease dramatically. When the carrier is a relatively short linear polylysine or PEI backbone, the backbone will have a molecular weight of less than 75,000, more preferably less than 30,000, and most preferably, less than 25,000. When the carrier is a relatively short branched polylysine or PEI backbone, however, the backbone will have a molecular weight less than 60,000, more preferably less than 55,000, and most preferably less than 50,000.

In some embodiments, the topical formulations are prepared in a solid form for ease of handling, transport, or storage. The solid form may be prepared by any method known in the art. Non-limiting examples of such methods include powder forms prepared by lyophilization, vacuum-drying, drum-drying or spray drying, with lyophilization and vacuum-drying being particularly preferred.

In some embodiments, the topical formulations of the invention comprises a nonionic surfactant. Generally, this invention contemplates the use of any non-ionic surfactant that has the ability to stabilize the therapeutic agent or cosmetic agent (e.g., botulinum toxin) and that is suitable for pharmaceutical use. In certain embodiments, the non-ionic surfactant is a polysorbate, non-limiting examples of which include polysorbate 20, polysorbate 40, polysorbate 60, and polysorbate 80. In other embodiments, the non-ionic surfactant is a sorbitan ester, non-limiting examples of which include Span 20, Span 60, Span 65, and Span 80. The invention also contemplates using Triton X-100 or NP-40 as the non-ionic surfactants. In addition, the invention contemplates embodiments in which combinations of different non-ionic surfactants are used in conjunction. In certain preferred embodiments, the non-ionic surfactant is selected from the group consisting of polysorbates, poloxamers, and sorbitans, with polysorbates and sorbitans being particularly preferred. In preferred embodiments, the concentration of the non-ionic surfactant is in the range of 0.005% to 0.5%, or in the range of 0.01% to 0.2%, or in the range of 0.02% to 0.1% or in the range of 0.05 to 0.08%. This invention also contemplates formulations where the concentration of the non-ionic surfactant is 0.01%, 0.02%, 0.03%, 0.04%, 0.05%, 0.06%, 0.07%, 0.08%, 0.09%, 0.10%, 0.11%, 0.12%, 0.13%, 0.14%, or 0.15%.

When the therapeutic or cosmetic active agent is a proteinaceous material, it is often desirable to stabilize the active agent before, during, or after lyophilization by including a non-reducing sugar in the topical composition. Generally speaking, the non-reducing sugar may be any sugar with a glass transition temperature above 60° C. In certain particularly preferred embodiments, the non-reducing sugar is a disaccharide, non-limiting examples of which include trehalose and sucrose. In other embodiments, the non-reducing sugar is a trisaccharide, a non-limiting example of which is raffinose. Generally, the concentration of the non-reducing sugar in the topical formulations of the invention are in the range of 10% to 40%, preferably 10% to 25%, more preferably 15% to 20%. In some preferred embodiments, the concentration of the non-reducing sugar is 10%, 11%, 12%, 13%, 14%, 15%, 16%, 17%, 18%, 19% or 20%.

In certain embodiments, the topical formulations of the invention comprise a bulking agent that makes it easier to handle lyophilized forms of the topical formulation. Preferably, the bulking agents crystallize under lyophilization conditions and do not mix well with the other excipients when in the solid state. Non-limiting examples of such bulking agents include sorbitol, mannitol, glycine, arginine, and histidine. The concentration of the bulking agent may be in the range of 1% to 10%, 2% to 6%, 3% to 5% or 4% to 4.5%. When a bulking agent is used, the concentration of the non-reducing sugar may be reduced from the 10% to 40% range to a range of 0.5% to 3.0%. Furthermore, in preferred embodiments, the ratio of the non-reducing sugar to the bulking agent is in the range of 0.07 to 2.0, preferably in the range of 0.4 to 0.6. Thus, by way of example only, the formulation may comprise mannitol as the bulking agent and trehalose as the non-reducing sugar, with mannitol present in a concentration range of 1.5% to 7.5% and trehalose present in a concentration range of 0.5% to 3.0%.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of the equivalents to which such claims are entitled.

1. A topical applicator for a paralytic agent, the topical applicator comprising:
a vial socket,
a housing having first and second open ends, wherein the first open end is releasably connected to the vial socket,
a needle hub mounted within the housing,
a needle having first and second piercing ends mounted in the needle hub,
a vial having a neck and a base, the vial being inserted in the vial socket, the vial being non-removably retained in the releasably connected vial socket,
a cartridge having a plunger sealing an open end thereof,
a septum located at an opposite end of the cartridge, and
an activation cap for causing the needle to penetrate a septum of the vial and the septum of the cartridge to permit transfer of components therebetween,
wherein the vial comprises a paralytic agent in solid form, and
wherein the cartridge comprises a diluent for reconstituting the paralytic agent.

2. The topical applicator according to claim 1, wherein the paralytic substance comprises a toxin from the group consisting of botulinum toxin, tetanus toxins, saxitoxins, and tetrodotoxin.

3. The topical applicator according to claim 2, wherein the paralytic agent comprises botulinum toxin.

4. The topical applicator according to claim 3, wherein the paralytic agent comprises botulinum toxin serotype A, B, C₁, D, E, F, or G.

5. The topical applicator according to claim 1, wherein the paralytic agent comprises botulinum toxin type A.

6. The topical applicator according to claim 1, wherein the diluent comprises a viscosity modifying agent.

7. The topical applicator according to claim 1, wherein the viscosity modifying agent is selected from the group consisting of a poloxamer, a polyalcohol, and hydroxyalkylcellulose.

8. The topical applicator according to claim 7, wherein the viscosity modifying agent is a poloxamer.

9. The applicator according to claim 8, wherein the poloxamer is selected from the group consisting of poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, and poloxamer 407.

10. The topical applicator according to claim 8, wherein the viscosity modifying agent is a polyalcohol.

11. The topical applicator according to claim 10, wherein the polyalcohol is polyethylene glycol.

12. The topical applicator according to claim 7, wherein the viscosity modifying agent is a hydroxylalkyl cellulose.

13. The topical applicator according to claim 12, wherein the viscosity modifying agent is selected from the group consisting of hydroxylpropylcellulose and hydroxypropyl methyl cellulose.

14. A topical applicator for a paralytic agent, the topical applicator comprising:
a housing having first and second open ends, wherein the first open end is configured to be releasably connected to a vial socket that is adapted to receive a vial,
a needle hub mounted within the housing,
a needle having first and second piercing ends mounted in the needle hub,
a cartridge having a plunger sealing an open end thereof,
a septum located at an opposite end of the cartridge, and
an activation cap for causing the needle to penetrate a septum of a vial held in the vial socket and the septum of the cartridge to permit transfer of components therebetween,
wherein the cartridge comprises a paralytic agent and a diluent for reconstituting the paralytic agent,
and wherein the needle hub fluidly connects a dispensing member that is attached to a distal end of the housing with the contents of the cartridge.

15. A method of topically applying a paralytic agent, the method comprising
pushing the activation cap of the topical applicator according to claim 1, to cause the needle to penetrate a septum of a vial held in the vial socket and the septum of the cartridge, thereby establishing a fluid connection,
pushing on the plunger to force the diluent into the vial and optionally shaking the vial, thereby forming a reconstituted paralytic composition,
withdrawing the plunger to cause the reconstituted paralytic composition to be drawn into the cartridge,
removing the vial socket and vial to expose a dispensing member, and
pushing on the plunger to cause the reconstituted paralytic composition to be dispense from the dispensing member onto an area on a patient in need of treatment.

16. The method according to claim 15, wherein the paralytic agent comprises botulinum toxin.

17. The method according to claim 16, wherein the botulinum toxin is botulinum toxin type A neurotoxin.

18. The method according to claim 15, wherein the diluent comprises water, saline, or a pharmaceutically acceptable buffer.

19. The method according to claim 18, wherein the diluent further comprises a viscosity modifying agent.

20. The method according to claim 19, wherein the viscosity modifying agent is a poloxamer.

21. The method according to claim 20, wherein the poloxamer is selected from the group consisting of poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, and poloxamer 407.

22. The method of claim 15, wherein reconstituted paralytic agent comprises a positively charged carrier.

23. The method according to claim 15, wherein the positively charged carrier comprises polylysine or a polyalkyleneimine.

24. The method according to claim 23, wherein the positively charged carrier has the amino acid sequence RKKRRQRRR-G-(K)₁₅-G-RKKRRQRRR (SEQ ID NO: 5).

25. The method according to claim 15, wherein the area on a patient in need of treatment is selected from the group consisting face, the axilla, the palms of the hands, the hands, the feet, the lower back, the neck, the leg, the groin, the arm, the elbow, the knee, the pelvis, the buttocks and the torso.

26. The method according to claim 25, wherein the area on the patient in need of treatment is the face, the reconstituted paralytic composition comprises botulinum toxin type A, and the reconstituted paralytic composition is used to reduce the appearance of wrinkles.

27. The method according to claim 26, wherein the wrinkles are selected from the group consisting of marionette lines, nasolabial lines, crows feet, brow furrows, glabellar lines, and combinations thereof.
